(19) United States
(12) Patent Application Publication
(10) Pub. No.: US 2026/0080988 A1
(43) Pub. Date: Mar. 19, 2026

(54) SYSTEMS AND METHODS FOR ANALYSIS OF MULTI-RESOLUTION IMAGES

(71) Applicant: PAIGE.AI, Inc., New York, NY (US)

(72) Inventors: Jeremy Daniel KUNZ, New York, NY (US); Adam CASSON, New York, NY (US); Fausto MILLETARÌ, Isnello (IT); George SHAIKOVSKI, London (GB); Theo GUGOIU, New York, NY (US); Siqi LIU, New York, NY (US)

(21) Appl. No.: 19/330,070

(22) Filed: Sep. 16, 2025

Related U.S. Application Data
(60) Provisional application No. 63/695,504, filed on Sep. 17, 2024.

(51) Int. Cl. G16H 10/60; G06F 16/532; G06F 16/583

(52) U.S. Cl. CPC G16H 10/60; G06F 16/532; G06F 16/583

(57) ABSTRACT

A system and method to analyze multi-resolution images. The system and method may receive at least one input that includes at least one natural language query from a user and a plurality of parameters. The system and method may generate one or more outputs by executing a machine learning algorithm to analyze the at least one multi-resolution image based on the at least one natural language query, wherein the one or more outputs comprise at least one natural language answer corresponding to the at least one natural language query and wherein the at least one natural language answer is provided for each of the plurality of parameters. The system and method may display, via a display interface, the at least one multi-resolution image and the one or more outputs.

### CROSS-REFERENCE TO RELATED APPLICATIONS

This application claims priority to U.S. Provisional Application No. 63/695,504, filed on Sep. 17, 2024, the entire disclosure of which is hereby incorporated by reference in its entirety.

### TECHNICAL FIELD

The present disclosure relates to systems, devices, and methods for analysis of multi-resolution images. Various embodiments of the present disclosure pertain generally to whole slide image (WSI) or digital pathology image analysis and related methods. More specifically, particular embodiments of the present disclosure relate to systems and methods for using Artificial Intelligence (AI) for analyzing image data such as WSI at different magnification levels and providing tailored feedback for each level.

### BACKGROUND

Pathology plays a crucial role in medicine by providing accurate diagnoses based on the microscopic analysis of tissue samples. In modern times, digital pathology and whole-slide imaging (WSI) has transformed the field, allowing pathologists to digitize and analyze tissue samples at high resolution at multiple magnification levels. This digital transformation offers numerous benefits, including improved workflow efficiency, remote access to images for consultation, and the potential for automated image analysis using artificial intelligence (AI) or machine learning algorithms.

However, despite the technological advances in digital pathology, the interpretation of complex WSI data remains a challenge for pathologists. In particular, pathologists need to constantly switch between magnification levels to examine different aspects of tissue morphology, which can be time-consuming and prone to human error.

Furthermore, current systems may not fully incorporate the ever-growing amount of metadata associated with cases, multiple resolutions, and patient-specific information to provide more comprehensive analysis. In an example, conventional approaches may not consider the entire clinical picture, and may have blind spots with regard to patient demographics, family history, community data, medical history, patient history, and other relevant data. Such gaps may inhibit an ability to make informed and/or accurate diagnostic decisions.

### SUMMARY OF THE DISCLOSURE

In an aspect of the present disclosure, described herein is a method for analyzing a multi-resolution image including: receiving at least one multi-resolution image; receiving at least one input that includes at least one natural language query from a user and a plurality of parameters; generating one or more outputs by executing a machine learning algorithm to analyze the at least one multi-resolution image based on the at least one natural language query, wherein the one or more outputs include at least one natural language answer corresponding to the at least one natural language query and wherein the at least one natural language answer is provided for each of the plurality of parameters; and displaying, via a display interface, the at least one multi-resolution image and the one or more outputs.

In some embodiments, the plurality of parameters are selected from the group consisting of a region of interest (ROI), one or more magnification levels, additional samples or images, patient information, or information tier. In at least one embodiments, the plurality of parameters are a ROI and a plurality of magnification levels. The plurality of parameters may be selected by a toggle switch displayed on a screen and the one or more outputs may have a visual indicator, for example a change in color, for each of the plurality of parameters.

In certain embodiments, the at least one input includes a language parameter, and the machine learning algorithm adjusts the natural language of the at least one natural language answer to correspond to the language parameter. The language parameter may be a patient-oriented mode or a teacher mode. In some embodiments, the machine learning algorithm to analyze the at least one multi-resolution image is provided one or more machine learning data sets that includes at least one of an analysis of a second multi-resolution image, a patient history, a family history, or a community health data.

In another aspect of the present disclosure, described herein is a method for analyzing a multi-resolution image including: receiving at least one multi-resolution image; receiving at least one input that includes at least one natural language query from a user; generating one or more outputs, by executing a machine learning algorithm to analyze the at least one multi-resolution image at a plurality of magnification levels based on the at least one natural language query, wherein the one or more outputs includes a plurality of natural language answers that correspond to the at least one natural language query, and wherein each answer of the plurality of natural language answers is specific to a respective level of the plurality of magnification levels; and displaying, via a display interface, the at least one multi-resolution image and the one or more outputs.

In some embodiments, the at least one input includes a region of interest. In another embodiment, the one or more outputs have a visual indicator for the respective magnification levels. In yet another embodiment, the machine learning algorithm is trained using different data sets for the respective magnification levels. In some embodiments, the machine learning algorithm to analyze the at least one multi-resolution image at a plurality of magnification levels is provided one or more machine learning data sets that includes at least one of an analysis of a second multi-resolution image, a patient history, a family history, or a community health data.

In yet another aspect, described herein is a system for multilevel magnification analysis of multi-resolution images including: a computer-readable storage medium storing instructions for generating and presenting a natural language answer corresponding to a natural language query from a user; a display interface; and one or more processors operatively connected to the computer-readable storage medium and the display interface, and configured to execute the instructions to perform operations including: receiving one or more multi-resolution images and the natural language query; automatically generating, by executing a machine learning algorithm to analyze the one or more multi-resolution image at a plurality of magnification levels, a natural language answer for the respective magnification levels that correspond to the natural language query; and causing the display interface to display the one or more multi-resolution images and the natural language answer.

In some embodiments, the natural language query includes a selection of one or more parameters and in at least one embodiment, the one or more parameters include a region of interest. The natural language answer may include one or more visual indicators, for example a change in color, to visually differentiate the natural language answer for the respective magnification levels. The one or more parameters includes one or more language parameters, and the machine learning algorithm may adjust the language of the natural language answer to correspond to the one or more language parameters.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary environment for training and/or using a machine learning model to analyze a multi-resolution image, according to one or more embodiments.

FIG. 2 depicts an example of a computing device, according to one or more embodiments.

FIGS. 3A-3B depict exemplary methods of training and using a machine learning model, according to one or more embodiments.

FIG. 3C depicts an exemplary display of a multi-resolution image with a selected region of interest and a natural language query, according to one or more embodiments.

FIG. 4 depicts an exemplary selection of answer parameters, according to one or more embodiments.

FIG. 5 depicts an exemplary display of one or more outputs generated by a machine learning model, according to one or more embodiments.

Notably, for simplicity and clarity of illustration, certain aspects of the figures depict the general configuration of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments.

### DETAILED DESCRIPTION

The foregoing general description is exemplary and explanatory only, and not restrictive of the disclosure. Other embodiments of the invention will be apparent to those skilled in the art from consideration of the specification and practice of the invention disclosed herein. It is intended that the specification and examples be considered as exemplary only.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially,” “approximately,” “about,” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” is, optionally, construed to mean “upon determining” or “in response to determining” depending on the context.

Terms like “provider,” “merchant,” “vendor,” or the like generally encompass an entity or person involved in providing, selling, and/or renting items to persons such as a seller, dealer, renter, merchant, vendor, or the like, as well as an agent or intermediary of such an entity or person. An “item” generally encompasses a good, service, or the like having ownership or other rights that may be transferred. As used herein, terms like “user” or “customer” generally encompasses any person or entity that may desire information, resolution of an issue, purchase of a product, or engage in any other type of interaction with a provider. The term “browser extension” may be used interchangeably with other terms like “program,” “electronic application,” or the like, and generally encompasses software that is configured to interact with, modify, override, supplement, or operate in conjunction with other software.

As used herein, a “machine learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine learning model may operate on an input linearly, in parallel, via a network (e.g., a neural network), or via any suitable configuration. By virtue of such training, a machine learning model is converted from an un-trained and un-specific model to a model that is unique to and specifically configured for the particular purpose for which it is trained. In an example, training of a machine learning model is analogous to a method of production in which the article produced is the trained model having unique characteristics by virtue of its particular training. Moreover, the result of training a machine learning model using particular training data and for a particular purpose results in a technical solution to an inherently technical problem.

The execution of the machine learning model may include deployment of one or more machine learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

The present disclosure generally provides for systems and methods for analyzing multi-resolution images through the use of a Question and Answer (Q&A) system. The development of a multilevel Q&A system in pathology aims, among other benefits and improvements, to bridge the gap between advanced AI capabilities and traditional diagnostic methodologies. A multilevel system, with AI assistance, may offer the ability to query an image and receive responses that are tailored to different levels of magnification and/or additional contextual data. Through an AI chat-based interface, a user can select regions of interest, specify the levels of information to include in their queries, and explore diverse data layers for a substantially more in-depth analysis. The system disclosed herein may recommend optimal magnification levels, provide detailed descriptions of cellular features, offer insights from cross-referenced slides or cases, and incorporate additional patient and community information. In some embodiments, the multi-resolution images are digital pathology slides. However, any suitable multi-resolution image may be used in various embodiments.

An exemplary environment for analyzing multi-resolution images may include, for example, an analysis system (the system). Various embodiments may include one or more of a user device, a data storage device, an imaging device, a display component etc. Such components may communicate over an electronic network. The system may generally comprise a computer-readable storage medium, one or more processors, a machine learning algorithm that includes a chat-based natural language Q&A component, and a display interface.

The system described herein may typically comprise a user-friendly display interface that allows a user to view the multi-resolution image, select answer parameters or a plurality of parameters, and input query information. In some embodiments, the system may include, for example, an image analysis algorithm, or any other modules etc. later described. The user may interact with the system through natural language queries, enabling intuitive communication and a more streamlined workflow. In some embodiments, the interface may provide recommendations on the standard analyses or queries typically conducted on specific image types, specimens, or stains.

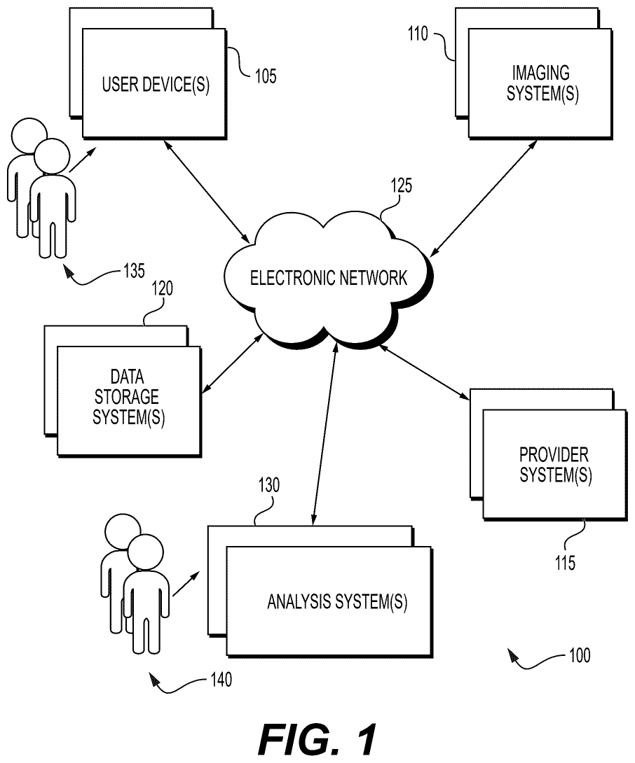

FIG. 1 depicts an exemplary environment 100 that may be utilized with techniques presented herein. One or more user device(s) 105, one or more imaging system(s) 110, one or more provider systems 115, one or more data storage system(s) 120, etc., may communicate across an electronic network 125. As will be discussed in further detail below, one or more analysis system(s) 130 may communicate with one or more of the other components of the environment 100 across electronic network 125. The one or more user device(s) 105 may be associated with a user 135, e.g., a user associated with one or more of generating, training, or tuning a machine learning model for a chat-based natural language Q&A, and/or generating, obtaining, or analyzing multi-resolution image data, e.g., using such a model.

In some embodiments, the components of the environment 100 are associated with a common entity, e.g., a financial institution, transaction processor, hospital network, medical practice, merchant, or the like. In some embodiments, one or more of the components of the environment 100 is associated with a different entity than another. The systems and devices of the environment 100 may communicate in any arrangement. As will be discussed herein, systems and/or devices of the environment 100 may communicate in order to one or more of generate, train, or use a machine learning model to analyze a multi-resolution image and developing a chat based natural language Q&A system, among other activities.

The user device 105 may be configured to enable the user 140 to access and/or interact with other systems in the environment 100. For example, the user device 105 may be a computer system such as, for example, a desktop computer, a mobile device, a tablet, etc. In some embodiments, the user device 105 may include one or more electronic application(s), e.g., a program, plugin, browser extension, etc., installed on a memory of the user device 105. In some embodiments, the electronic application(s) may be associated with one or more of the other components in the environment 100. For example, the electronic application(s) may include one or more of a portal or interface for interacting with another element of the environment 100, a tool for generating, tuning, or interacting with the machine-learning model, or a program or interface for analyzing multi-resolution images, e.g., that is configured to utilize or interact with a trained model, etc.

The imaging system 110 may include any suitable device for capturing medical images that may form the basis of a multi-resolution image. In an example, an imaging system 110 may include a camera, sensor, or the like, and may operate with or be included in a device such as, for example, a microscope. In another example, the imaging system 110 may be integrated into or operate with a medical imaging device, such as an X-Ray device, ultrasound device, radiographic imaging device, etc. In some embodiments, images captured by the imaging system 110 may be stored, e.g., via the data storage system 120, the provider system 115, or the like, or may be used to generate multi-resolution images. In some instances, an imaging system 110 may be configured to capture a multi-resolution image, e.g., capture multiple resolutions or levels of detail of a region of interest in parallel.

The provider system 115, which may include a server system, an electronic data system, computer-readable memory such as a hard drive, flash drive, disk, etc., may include and/or interact with an application programming interface for exchanging data to other systems, e.g., one or more of the other components of the environment 100. The provider system 115 may include and/or act as a repository or source for patient data, e.g., data pertaining to a patient's health information, data segmented into a particular case, a particular slide, etc.

The data storage system 120 may include a server system, an electronic data system, computer-readable memory such as a hard drive, flash drive, disk, etc. In some embodiments, the data storage system 120 may include and/or interact with an application programming interface for exchanging data to other systems, e.g., one or more of the other components of the environment 100. The data storage system 120 may include and/or act as a repository or source for image data.

In various embodiments, the electronic network 125 may be a wide area network (“WAN”), a local area network (“LAN”), personal area network (“PAN”), or the like. In some embodiments, electronic network 125 includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

As discussed in further detail below, the analysis system 130 may one or more of (i) generate, store, train, or use a machine learning model configured to analyze multi-resolution images through the use of a Q&A system. The analysis system 130 may include a machine learning model and/or instructions associated with the machine learning model, e.g., instructions for generating a machine learning model, training the machine learning model, using the machine learning model etc. The analysis system 130 may include instructions for retrieving image data, adjusting image data, e.g., based on the output of the machine learning model, and/or operating the user device 105 to output image data, e.g., as adjusted based on the machine learning model. The analysis system 130 may include training data, e.g., multi-resolution image data sets, question-answer pairs, image-caption pairs, image segmentation masks, fine-grained image categorization labels, and natural language descriptions of images. Additional data sets may include medical data sets, e.g. patient history, family history, community health data, diagnosis trends, as well as associated labels, e.g. ROIs, annotations, features, findings, measurements, patterns, etc.

In certain embodiments, a training process may encompass the analysis of a multi-resolution image dataset at various magnification levels, e.g., relative to known information regarding the subject of the multi-resolution images in the dataset. For example, each training sample may include a multi-resolution image along with labeling that is relevant at different resolutions. Additionally, different batches of training data may be configured to target different scenarios including specific targeted diagnoses, patient demographics, or clinical scenarios. For example, one batch of training data may be configured to target the diagnosis of lesions from a CT scan while another batch may be configured to target the diagnosis of diabetic retinopathy from fundus photography images.

In some embodiments, a system or device other than the analysis system 130 is used to generate and/or train the machine learning model. For example, such a system may include instructions for generating the machine learning model, the training data and ground truth, and/or instructions for training the machine learning model. A resulting trained-machine learning model may then be provided to the analysis system 130.

Generally, a machine learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable. In unsupervised learning, patterns, correlations, and/or clusters of input samples may be used to determine one or more metrics or features of the samples usable to differentiate between related subsets of the samples. In semi-supervised learning, unsupervised and supervised approaches may be combined.

Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training and/or used to validate the trained machine learning model, e.g., compare the output of the trained model with the ground truth for that portion of the training data to evaluate an accuracy of the trained model. The training of the machine learning model may be configured to cause the machine learning model to learn associations between training data and ground truth data, such that the trained machine learning model is configured to determine one or more outputs in response to the at least one input based on the learned associations. Particular selection and/or application of training data, such as discussed in various embodiments of this disclosure, may inhibit or reduce impact of concerns such as biasing (e.g., via selection, truncation, or the like), overfitting, under-fitting, etc.

In some instances, training using one set or type of data may be used or adapted to another set of data. For example, a modal initially trained on one data set may require less samples or time to train on a second data set. In another example, initial training may result in a base model that may be tuned with an additional data set so as to form a particularized model specific to circumstances of the additional data set.

In various embodiments, the variables of a machine learning model may be interrelated in any suitable arrangement in order to generate the output. For example, in some embodiments, the machine learning model may include image-processing architecture that is configured to identify, isolate, and/or extract features, geometry, and or structure in one or more of the medical imaging data and/or the non-optical in vivo image data. For example, the machine learning model may include one or more convolutional neural network (“CNN”) configured to identify features in the image data, and may include further architecture, e.g., a connected layer, neural network, etc., configured to determine a relationship between the identified features in order to determine a location in the image data.

Various features may be included or used with any suitable machine learning model. For instance, a model may be configured to receive and or determine a relative positioning of data or portions of data in samples (e.g., position of words in a sentence, location of pixels in an image, etc.), and use such positions as a portion of the input to the model. In another instance, a model configured to utilize attention may be configured to weigh, determine, or the like how different samples or portions of samples impact the output of the model, and may incorporate such data into the training process. An example of a model that utilizes information on relative positioning and attention is a transformer model. One implementation incorporating a transformer is a large language model. Transformers and other suitable models have been used for multi-modal input, e.g., a model that is configured to use and process input of different modalities (a combination of or selection from one or more of text, audio, video, structured or unstructured data, etc.).

As described herein, any suitable type of machine learning model or combination of machine learning models may be used. Operations conducted by one model in some embodiments may be distributed amongst a plurality of models in other embodiments, or vice versa.

Although depicted as separate components in FIG. 1, it should be understood that a component or portion of a component in the environment 100 may, in some embodiments, be integrated with or incorporated into one or more other components. For example, a portion of the analysis system 130 may be integrated into the user device 105 or the like. In another example, the analysis system 130 may be integrated with the data storage system 120. In some embodiments, operations or aspects of one or more of the components discussed above may be distributed amongst one or more other components. Any suitable arrangement and/or integration of the various systems and devices of the environment 100 may be used.

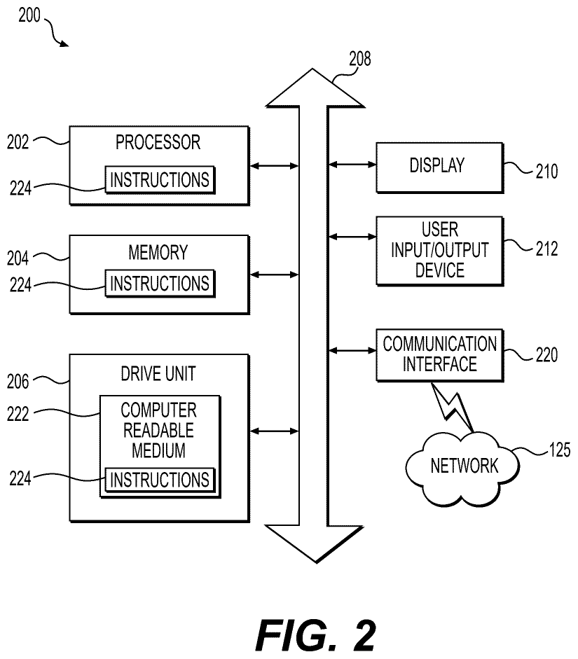

FIG. 2 is a simplified functional block diagram of a computer 200 that may be configured as a device for executing the machine learning methods, according to exemplary embodiments of the present disclosure. For example, the computer may be configured as the analysis system 130 and/or another system according to exemplary embodiments of this disclosure. In various embodiments, any of the systems herein may be a computer 200 including, for example, a data communication interface 220 for packet data communication. The computer 200 also may include a central processing unit (“CPU”) 202, in the form of one or more processors, for executing program instructions.

The computer 200 may include an internal communication bus 208, and a storage unit 206 (such as ROM, HDD, SDD, etc.) that may store data on a computer readable medium 222, although the computer 200 may receive programming and data via network communications. The computer 200 may also have a memory 204 (such as RAM) storing instructions 224 for executing techniques presented herein, although the instructions 224 may be stored temporarily or permanently within other modules of computer 200 (e.g., processor 202 and/or computer readable medium 222). The computer 200 also may include input and output ports 212 and/or a display 210 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments may be applicable to any environment, such as a desktop or laptop computer, an automobile entertainment system, a home entertainment system, etc. Also, the disclosed embodiments may be applicable to any type of Internet protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

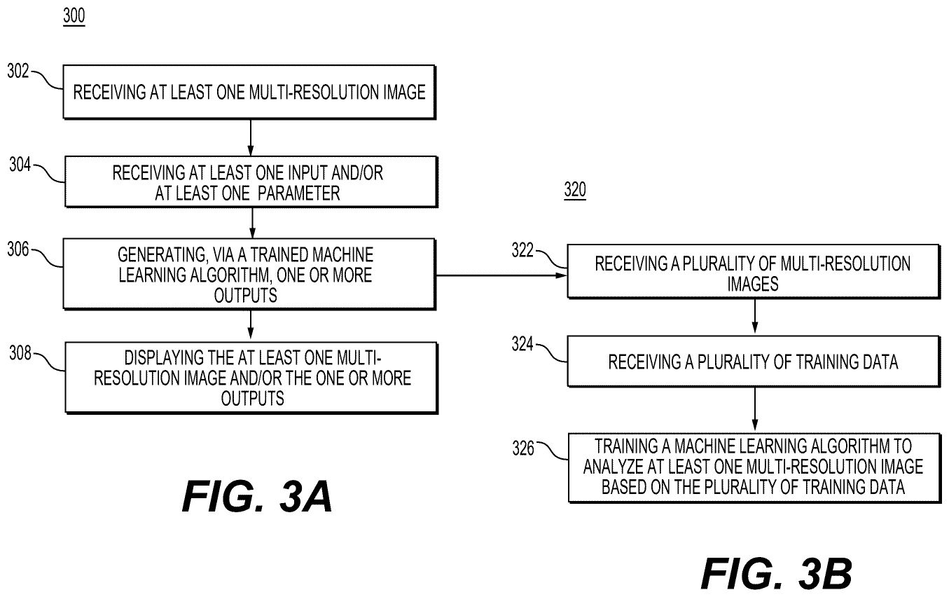

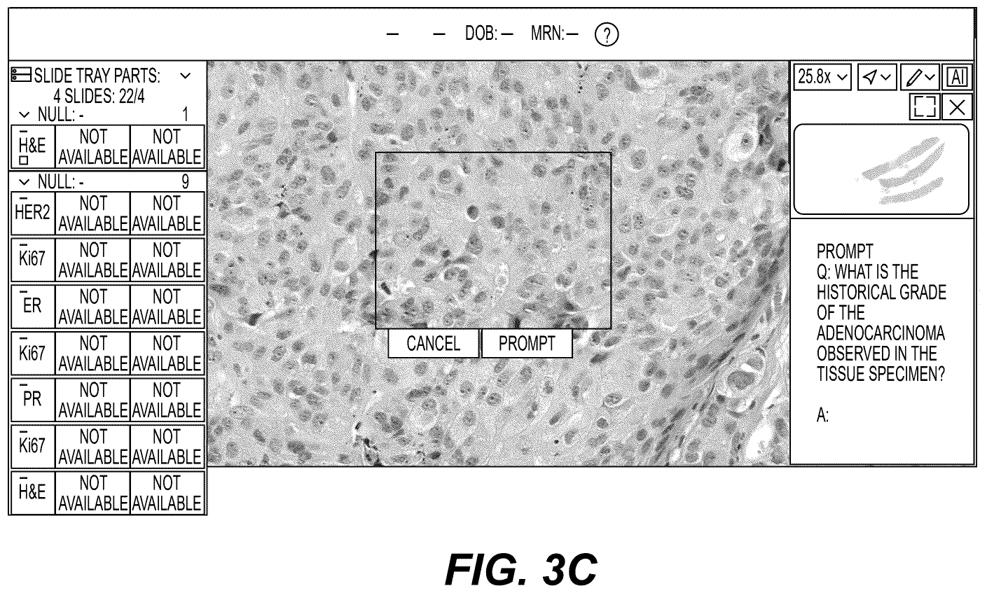

FIGS. 3A-3B depict exemplary methods 300, 320 of using and training a machine learning algorithm, respectively. An initial stage of the present disclosure may generally involve receiving a multi-resolution image (step 302). In some embodiments, the initial stage may involve receiving one or more multi-resolution images or a plurality of multi-resolution images. The multi-resolution image may be imported through a provider system 110 or data storage system 120 associated with a hospital network or the like, manually uploaded, or obtained via any suitable technique. In at least some embodiments, the multi-resolution image may be transmitted through a network 125. The multi-resolution image may include, be associated with, or accompanied by details pertaining to, for example, the type of specimen used, how the specimen and/or multi-resolution image was prepared, what stains or histological processes were used, and/or details pertaining to patient health information (PHI) including, but not limited to, family history, patient history, previous pathology reports, and/or other health care services provided, e.g., as may be accessed via the provider system 115. These details may be received in the same manner as the multi-resolution image or through other means such as through manual input.

The analysis system 130 may perform an initial analysis, such as through the execution of an image analysis algorithm or a machine learning algorithm, of the multi-resolution image to provide preliminary recommendations or guidance, and/or preliminary identification of structures or artifacts. In some embodiments, the multi-resolution image may undergo a quality control and assurance procedure to ensure sufficient resolution and/or verify the accuracy of reported information such as matching the identified staining method with the reported staining method. The analysis system 130 may use the initial analysis and/or quality control and assurance procedure to identify and report any inaccuracies or discrepancies in the image, or exclude magnification levels that are not supported by the resolution of the image.

Referring to FIG. 3C, a user 135 may generally provide one or more inputs or at least one input, e.g., via a graphical user interface. The at least one input may include, for example, one or more of an identification of an ROI, e.g., by selecting a region of an image using the interface, a natural language query or one or more natural language queries, a selection from one or more predetermined or pre-generated queries, etc. Such queries may be directed to different diagnoses, identification of certain structures, or other diagnostic information. In an example, the user 135 may, e.g., via the interface, make selections to include or exclude one or more predetermined query, e.g., instead of or in addition to providing other input such as the options discussed above.

In some embodiments, the at least one input may include a selection or identification of a parameter or plurality of parameters (step 304 of FIG. 3A). The parameters may include a region of interest (ROI), one or more magnification levels, different specimens or slides, patient information, and information tiers that include different levels of information or databases such as a patient history or community health data. In an example, a natural language query may include text indicative of a particular magnification level, of a particular feature in an image usable to determine an ROI, or the like.

A user may designate a ROI through a multitude of selection methods. For example, a user may click on a specified area displayed on a screen, utilize a tool to encircle and highlight a desired region, and utilize a drag-and-drop function to outline a desired region, or specify a directed area of the slide (e.g. top of slide). In some embodiments, a ROI may be highlighted during the initial analysis of the multi-resolution image, e.g., based on a preliminary image analysis to identify one or more features, or the like.

In some embodiments, the user 135 may specify certain magnification levels such as 80×, 40×, 20×, 10×, 5×, 2×, and/or 1×. In addition to setting magnification levels, the user 135 may have additional methods for selecting magnification levels that are more tailored to their analytical needs and preferences. These additional methods may include, for example, setting a slide tool, inputting numerical values, or employing a zoom-in function. In an exemplary embodiment, a user may select a plurality of magnification levels, as discussed in further detail below.

In addition to parameters related to the multi-resolution image, the user 135 may select parameters that include or identify additional images or specimens. For example, the user 135 may include or identify all images related to a specific specimen, all images related to a patient, or all images related to a specific condition. These additional images may be prepared in the same way as the initial image, or may be prepared in a different way such as by using a different stain. This functionality supports in-depth investigations into tissue morphology, staining patterns, or cellular structures across different specimens and patient cases, enhancing the depth and breadth of insights derived from the analysis process.

The user 135 may want to include additional patient information such as age, medical history, prior diagnoses, travel history, and/or medication information. Such information may be usable to identify additional causations to why a certain feature is found or add additional context. The additional patient context may enable identification of possible correlations and causative factors behind observed features or provide contextual relevance to the analysis. In certain embodiments, the user 135 may further select various informational databases or “levels of data” such as family history or community trends to provide even greater insight or to allow for the identification of patterns or trends in the data. The interface of the analysis system 130 may further enable the selection of additional variables for analysis, such as the meaning of a diagnosis at different lengths of time or factoring an unknown variable. For example, in a case where an exposure is unknown, the system may provide a likelihood of diagnosis data at different lengths of time since exposure.

The display interface may use various methods to allow the user 135 to select parameters. The user 135 may select parameters as part of the natural language query or the system may automatically select toggles based off of the natural language query. For example, the user 135 may specify which magnification levels to include as part of the natural language query. In some embodiments, the machine learning algorithm may interpret the natural language query to indicate certain parameters, or the algorithm may automatically select parameters based on the user 135's information requirements.

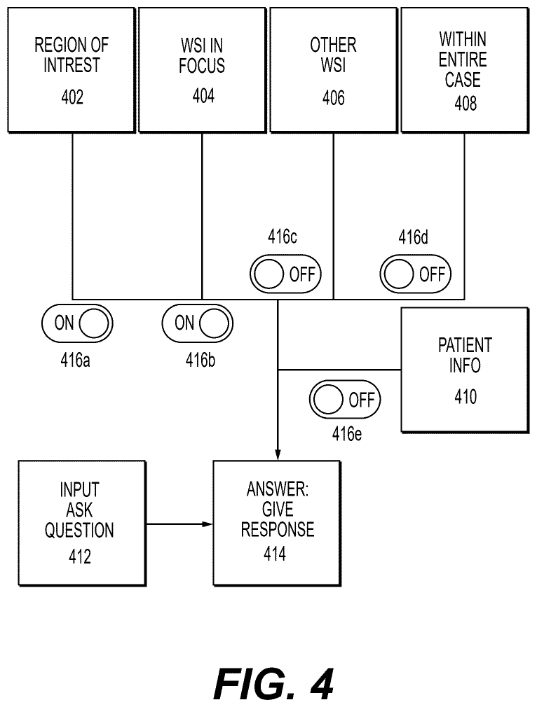

As shown in FIG. 4, the user 135 may use virtual toggle switches 416a-e displayed on the display interface to adjust and customize the parameters. The display interface may have toggle switches associated with any suitable parameters, such as a region of interest 402 and toggle switch 416a, WSI(s) in focus 404 and toggle switch 416b, other WSI(s) and toggle switch 416c, within entire case 408 and toggle switch 416d, patient information 410 and toggle switch 416e, etc. For example, the display interface may have a toggle switch to affirmatively include or exclude different magnification levels from the image analysis (not displayed). A user may positively assert certain levels or negatively exclude others based on the diagnostic requirements, providing flexibility and control over the depth of answer. In another example, an additional toggle switch may be selected to cause the analysis system 130 to perform a search for other images, e.g., radiology images, and pull in such images in a manner similar to the procedure discussed above. In certain embodiments, the initial analysis stage may include an indication of which toggles should be turned on and off. In some embodiments, the initial analysis stage may modify which parameters may be toggled on and off. A response 414 may be given based on which toggle switches are on or off, an input ask question 412, etc.

In some embodiments, the system may implement various visual indicators to assist a user in distinguishing information provided for each of the plurality of parameters. Visual indicators may include any attribute that contributes to visual differentiation. Examples of visual indicators may include color-coding, changes in font, bolding of text, different text sizes, different text colors, different background colors, or placing information in different boxes. The visual indicators may aid a user in interpreting responses and understanding the context of the at least one answer.

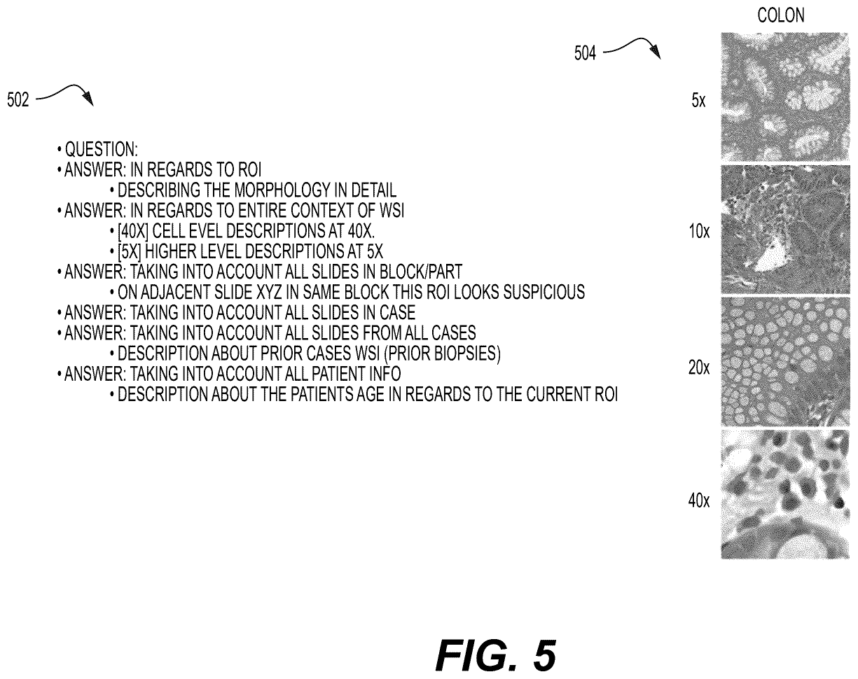

At step 306 of FIG. 3A, a machine learning algorithm may analyze the at least one multi-resolution image, the at least one input, and/or the at least one parameter to generate one or more output. Referring to FIG. 5, once the user 135 inputs the at least one natural language query and one or more parameters are selected, the analysis system 130 may generate different information based on the analysis of the multi-resolution image and provide a natural language answer 502 that is tailored to the selected parameters. In some embodiments, the analysis system 130 may analyze the multi-resolution at multiple magnifications 504 and may provide a natural language answer for the respective magnification levels. For example, as depicted in FIG. 5, if a user requests information about a specific cell morphology and chooses different magnifications as the parameters, the system may generally generate a natural language answer, which provides details about the requested morphology at each specified magnification level. This may include providing the number of cells with the specified morphology at a low magnification level and a more detailed characterization of the morphology at a higher magnification level. Annotations or other visual indications may also be applied to various magnification levels of the multi-resolution image.

In another example, the machine-learning model may be configured to consider multiple levels of resolution when generating a response, such that a response to a query pertaining to a particular resolution may include or account for information available to the model at a different resolution. In a further example, the machine-learning model may be configured to consider other information included or identified with the input. For instance, a response to a query pertaining to a particular slide or slides may include or account for the other information, such as information or data included or indicated by a Radiographic image, by patient data, by demographic data, etc. In other words, in some embodiments, the machine-learning model may be configured to accept a variety of amounts and modalities of input, any may consider any and all such data, even when considering a query pertaining to a single image, a single modality of data, or the like. In an exemplary use case, the machine-learning model may have been trained on a variety of modalities of data, e.g., image slides, MRI images, etc., and thus may be trained to learn associations between such data that may not be readily apparent by consideration of any one modality in isolation. In some embodiments, an ROI specified with the input may be at any magnification level, and a description of morphology included in a response may be specifically generated for each magnification level, with regard to the patient as a whole, with regard to a case or condition, or any other level of granularity of analysis.

The analysis system 130 may allow a user to select a variety of language parameters that may change or customize the generated natural language output. The language parameter may change the language used to generate the one or more outputs and/or change the level of sophistication used when generating the one or more outputs. In natural language processing (NLP), different domains, vernaculars, etc., may be considered different output targets that may be specified or selected between. As would be understood by one of ordinary skill in the art, an output of a machine learning model (e.g., an output vector) may be converted into natural language via an NPL process. By selecting an NLP process with a particular domain, the output may be used to generate natural language having desired characteristics. In some embodiments, the system may have three language parameters, a normal mode, teacher mode, and a patient-oriented mode, e.g., each corresponding to a respective natural language domain. However, it should be understood that the system may have any number of possible language parameters, customized to the needs of individual users. A teacher mode changes the generated natural language output to include additional explanatory slides. This mode may be desirable for education settings to facilitate training and professional development. The analysis system 130 may be configured to deliver detailed educational responses suitable for training and professional development. In a patient-oriented mode, the analysis system 130 may cause the generated natural language output to include explanations of the analysis using less complex language (e.g. layman's terms) or may exclude certain parts of the analysis that require greater medical proficiency. This may promote patient access and understanding of diagnostic findings.

In some embodiments, the analysis system 130 described herein may comprise additional functions, such as the ability to request additional images, specimens, and/or additional information. For example, the analysis system 130 may recommend a patient undergo additional testing to confirm a diagnosis or ask for verification of the presence of a substance. In some embodiments, the analysis system 130 may indicate the confidence levels of what it is reporting or create a screenshot with the ability to automatically redact protected patient information.

It should be understood that steps of one or more of the foregoing method described herein may be combined in certain embodiments. Furthermore, in certain embodiments, fewer than all of the steps of a method described herein may be performed and/or additional steps not described herein may be performed. Moreover, the steps described herein need not necessarily be performed in the exact order presented.

An initial or first step generally involves receiving at least one multi-resolution image. In some embodiments, this step includes performing a preliminary image analysis and a quality control and assurance process. In certain embodiments, this step may further comprises providing recommendation or guidance to a user 135. A second step generally involves receiving input from a user. The input generally comprises at least one natural language query. In some embodiments, the input may include the selection of a plurality of parameters, such as selecting certain magnification levels. In some embodiments, this input may include, e.g., may only include, receiving an instruction to perform a default, e.g., a predetermined query.

A third step generally involves executing a machine learning algorithm to analyze the at least one multi-resolution image based on the at least one natural language query. A fourth step generally involves providing a natural language answer that corresponds to the at least one natural language query. In some embodiments, the natural language answer is tailored to each of the plurality of parameters. In a fifth step, the analysis system 130 may display the multi-resolution image and generated output via a display interface, allowing a user to visualize and interpret the information presented (step 308 of FIG. 3A). In a sixth step, the analysis system 130 may be configured to receive follow-up input from the user 135. Such further input may be used to, for example, refine or replace the initial input and iterate the process. In an example, a previous response or responses may be combined with the further input, e.g., so that subsequent iterations of operation of the machine-learning model may account for information included in the previous responses.

As discussed herein, the machine learning algorithm may be trained, e.g., via method 320 of FIG. 3B. At step 322, a plurality of multi-resolution images may be received. At step 324, a plurality of training data may be received. As discussed herein, the training data may include multi-resolution image data sets, question-answer pairs, image-caption pairs, image segmentation masks, fine-grained image categorization labels, and natural language descriptions of images. Additional training data sets may include medical data sets, e.g. patient history, family history, community health data, diagnosis trends, as well as associated labels, e.g. ROIs, annotations, features, findings, measurements, patterns, etc. At step 326, the machine learning algorithm may be trained based on the plurality of multi-resolution images and the plurality of training data to analyze at least one multi-resolution image to generate an output.

The system and method described herein may include a device comprising a central processing unit (CPU). The CPU may be any type of processing device including, for example, any type of special purpose or a general-purpose microprocessor device. As will be appreciated by persons skilled in the relevant art, the CPU also may be a single processor in a multi-core/multiprocessor system, such system operating alone, or in a cluster of computing devices operating in a cluster or server farm. The CPU may be connected to a data communication infrastructure, for example a bus, message queue, network, or multi-core message-passing scheme.

The device may further include a main memory, for, random access memory (RAM), and may also include a secondary memory. Secondary memory, e.g., a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, a secondary memory may include similar means for allowing computer programs or other instructions to be loaded into device. Examples of such means may include a program cartridge and cartridge interface (such as that found in video game devices), a removable memory chip (such as an EPROM or PROM) and associated socket, and other removable storage units and interfaces, which allow software and data to be transferred from a removable storage unit to device.

The device also may include a communications interface (“COM”) that allows software and data to be transferred between the device and external devices. The communications interface may include a model, a network interface (such as an Ethernet card), a communications, a PCMCIA slot and card, or the like. Software and data transferred via the communications interface may in the form of signals, which may be electronic, electromagnetic, optical or other signals capable of being received by communications interface. These signals may be provided to communications interface via a communications path of device, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. Throughout this disclosure, references to components or modules generally refer to items that logically can be grouped together to perform a function or group of related functions. Components and modules may be implemented in software, hardware or a combination of software and hardware.

The tools, modules, and functions described above may be performed by one or more processors. “Storage” type media may include any or all of the tangible memory of the computers, processors, or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

The foregoing general description is exemplary and explanatory only, and not restrictive of the disclosure. Other techniques of the invention will be apparent to those skilled in the art from consideration of the specification and practice of the invention disclosed herein. It is intended that the specification and examples to be considered as exemplary only.

What is claimed is:

1. A method for analyzing a multi-resolution image, comprising:
receiving at least one multi-resolution image;
receiving at least one input that includes at least one natural language query from a user and a plurality of parameters;
generating one or more outputs by executing a machine learning algorithm to analyze the at least one multi-resolution image based on the at least one natural language query, wherein the one or more outputs comprise at least one natural language answer corresponding to the at least one natural language query and wherein the at least one natural language answer is provided for each of the plurality of parameters; and
displaying, via a display interface, the at least one multi-resolution image and the one or more outputs.

2. The method of claim 1, wherein the plurality of parameters are selected from the group consisting of a region of interest (ROI), one or more magnification levels, additional samples or images, patient information, or an information tier.

3. The method of claim 2, wherein the plurality of parameters include a ROI and a plurality of magnification levels.

4. The method of claim 2, wherein the plurality of parameters are selected by a toggle switch displayed on a screen.

5. The method of claim 1, wherein the one or more outputs have a visual indicator for each of the plurality of parameters.

6. The method of claim 5, wherein the visual indicator is a change in color.

7. The method of claim 1, wherein the at least one input includes a language parameter, and wherein the machine learning algorithm adjusts the natural language of the at least one natural language answer to correspond to the language parameter.

8. The method of claim 7, wherein the language parameter is a patient-oriented mode or a teacher mode.

9. The method of claim 1, wherein the machine learning algorithm used to analyze the at least one multi-resolution image is provided one or more machine learning data sets that includes at least one of an analysis of a second multi-resolution image, a patient history, a family history, or community health data.

10. A method for analyzing a multi-resolution image comprising:
receiving at least one multi-resolution image;
receiving at least one input that includes at least one natural language query from a user;
generating one or more outputs, by executing a machine learning algorithm to analyze the at least one multi-resolution image at a plurality of magnification levels based on the at least one natural language query, wherein the one or more outputs comprises a plurality of natural language answers that correspond to the at least one natural language query, and wherein each answer of the plurality of natural language answers is specific to a respective level of the plurality of magnification levels; and
displaying, via a display interface, the at least one multi-resolution image and the one or more outputs.

11. The method of claim 10, wherein the at least one input includes a region of interest.

12. The method of claim 10, wherein the one or more outputs have a visual indicator for the respective magnification levels.

13. The method of claim 10, wherein the machine learning algorithm has been trained using different data sets for the respective magnification levels.

14. The method of claim 10, wherein the machine learning algorithm used to analyze the at least one multi-resolution image at a plurality of magnification levels is provided one or more machine learning data sets that includes at least one of an analysis of a second multi-resolution image, a patient history, a family history, or community health data.

15. A system for multilevel magnification analysis of multi-resolution images comprising:
a computer-readable storage medium storing instructions for generating and presenting a natural language answer corresponding to a natural language query from a user;
a display interface; and
one or more processors operatively connected to the computer-readable storage medium and the display interface, and configured to execute the instructions to perform operations including:
receiving one or more multi-resolution images and the natural language query;
automatically generating, by executing a machine learning algorithm to analyze the one or more multi-resolution image at a plurality of magnification levels, a natural language answer for the respective magnification levels that correspond to the natural language query; and
causing the display interface to display the one or more multi-resolution images and the natural language answer.

16. The system of claim 15, wherein the natural language query comprises a selection of one or more parameters.

17. The system of claim 16, wherein the one or more parameters comprise a region of interest.

18. The system of claim 16, wherein the one or more parameters comprises one or more language parameters, and wherein the machine learning algorithm adjusts the language of the natural language answer to correspond to the one or more language parameters.

19. The system of claim 15, wherein the natural language answer comprises one or more visual indicators to visually differentiate the natural language answer for the respective magnification levels.

20. The system of claim 19, wherein the one or more visual indicators is a change in color.
